(19) United States
(12) Patent Application Publication
(10) Pub. No.: US 2016/0256221 A1
(43) Pub. Date: Sep. 8, 2016

(54) ANESTHESIA COVER SYSTEM

(71) Applicant: Donald L. Smith, Louisville, KY (US)

(72) Inventor: Donald L. Smith, Louisville, KY (US)

(21) Appl. No.: 14/639,175

(22) Filed: Mar. 5, 2015

(51) Int. Cl. A61B 19/08; A61M 16/01

(52) U.S. Cl. CPC A61B 19/081; A61M 16/01; A61B 19/088; A61M 2205/0238

(57) ABSTRACT

The present disclosure is directed to barriers for use with anesthesia machines. In certain aspects, the present disclosure provides unique medical devices, systems, and methods that can effectively provide a clean touch surface on medical equipment without inhibiting function of the medical equipment. In accordance with certain embodiments, such medical devices are configured as barriers adapted for placement over a portion of an anesthesia machine. Accordingly, in one embodiment the present disclosure demonstrates a system for providing clean touch surfaces on anesthesia equipment, the system including: one or more touchscreen barriers configured to securely adhere to and substantially cover the touchscreen of an anesthesia machine, and one or more control member barriers configured to securely adhere to and substantially cover a control member portion of an anesthesia machine.

### BACKGROUND

During medical procedures in which an anesthetic is required, the anesthesiologist generally uses an anesthesia machine to facilitate, control, and monitor the anesthesia process. An anesthesia machine is configured to provide an accurate and continuous supply of an anesthetic to the patient. During the procedure the anesthesia machine allows the anesthesiologist to monitor and manipulate the concentration of the inhalational agent (e.g. Desflurane, Sevoflurane, Isoflurane, Nitrous Oxide) mixed with a medical gas (e.g. oxygen) and the flow rate of the anesthetic. It is necessary for the anesthesiologist to touch and manipulate the machine during the procedure.

Recently there has been an increased demand for standardization of operating room procedures to minimize the risk of complications, including bacterial transmission. Among the new guidelines being developed is a requirement for physicians to wash their hands every time between touching a patient and touching a piece of durable equipment (e.g. an anesthesia machine). For anesthesiologists working in an operating room, this standard is impractical because they must repeatedly interact with both the patient and the anesthesia machine and do not have the opportunity to safely leave their stations and wash their hands. There is, therefore, a need for a system that would allow an anesthesiologist to safely interact with the patient and the anesthesia machine while complying with modern standardized practices.

### SUMMARY

In certain aspects, the present disclosure provides unique medical devices that can effectively provide a clean touch surface on medical equipment without inhibiting function of the medical equipment. In accordance with certain embodiments, such medical devices are configured as barriers adapted for placement over a portion of a piece of durable medical equipment (e.g an anesthesia machine). Accordingly, in one embodiment the present disclosure demonstrates a system for providing clean touch surfaces on anesthesia equipment, the system including: one or more touchscreen barriers configured to securely adhere to and substantially cover the touchscreen of an anesthesia machine, and one or more control member barriers configured to securely adhere to and substantially cover a control member portion of an anesthesia machine. In certain embodiments the barriers are configured so as to be removable after use. In some forms the disclosure provides for electroconductive touchscreen covers. In some forms the control member barrier is configured to cover a vaporizor control, a ventilator control, and/or a syringe pump control.

In another embodiment, the disclosure provides a method of using an anesthesia machine having one or more control members. Such method comprises applying one or more barriers configured to securely adhere to and substantially cover the control member portion of the anesthesia machine so as to provide a clean touch surface while preserving the functionality of the underlying control member. In certain embodiments the method also includes the step of removing the barrier after use of the control member.

In another inventive aspect the disclosure provides a barrier for use with an anesthesia machine, the barrier comprising an inner surface configured to contact a portion of the anesthesia machine, and an outer surface configured to be contacted by a user of the anesthesia machine such that the barrier allows full functionality of the covered portion of the anesthesia machine while preventing user contact with the machine. In certain embodiments the barrier comprises a conformable material.

Additional embodiments, as well as features and advantages of the disclosed embodiments, will be apparent from the description herein.

### BRIEF DESCRIPTION OF THE DRAWINGS

FIG. 1 illustrates one embodiment of a system for providing clean touch surfaces on anesthesia equipment.

FIG. 2a illustrates one embodiment of a barrier according the present disclosure.

FIG. 2b illustrates the barrier of FIG. 2a attached to a control member.

FIG. 3a illustrates another embodiment of a barrier according the present disclosure.

FIG. 3b illustrates the barrier of FIG. 3a attached to a control member.

FIG. 4a illustrates one embodiment of a barrier according the present disclosure.

FIG. 4b illustrates the barrier of FIG. 4a attached to a control member.

FIG. 4c illustrates another embodiment of a barrier according the present disclosure.

FIG. 5 illustrates another embodiment of a barrier according the present disclosure.

FIG. 6a illustrates one embodiment of a touchscreen barrier according to the present disclosure.

FIG. 6b illustrates another embodiment of a touchscreen barrier according to the present disclosure.

### DESCRIPTION OF THE SELECTED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, and alterations and modifications in the illustrated device, and further applications of the principles of the disclosure as illustrated therein are herein contemplated as would normally occur to one skilled in the art to which the disclosure relates.

In some forms the present disclosure provides systems, methods, kits, and/or devices for providing a barrier between the anesthesia machine and an operator (e.g. anesthesiologist). For example, in certain embodiments the present disclosure provides a barrier configured for placement over a portion of the anesthesia device commonly manipulated by the operator. In accordance with certain embodiments, the present disclosure provides one or more barriers configured to cover control members, touchscreens, and/or machine surfaces, while allowing for full functionality of the anesthesia machine. In some forms the barriers provided by the present disclosure are designed to be disposable such that the barriers can be placed and removed with each procedure. The present disclosure therefore provides a low cost solution to the problems associated with anesthesia machine operation.

In use, the disclosed barriers are configured to cover common touch surfaces of an anesthesia machine thus ensuring clean touch surfaces during a procedure. In some forms, the barriers are configured to be disposable and may be replaced prior to each use of the anesthesia machine.

In certain embodiments the present disclosure provides a barrier for use with a component of an anesthesia machine. In some forms, the disclosure provides for a barrier comprising a layer of substantially transparent material. In certain embodiments the barrier has dimensions comparable to the component of the anesthesia machine to be covered. In accordance with certain inventive variants the disclosed barriers may comprise a polymer such as for example: polyethylene, polyesters, polycarbonates, polyamides, ethylene vinyl acetate copolymer, polyvinyl chloride, polysulphones, cellulose acetate, and/or copolymers or derivatives thereof.

In accordance with certain inventive variants the disclosed barrier is removably fixable to a control surface of an anesthesia machine. In some modes, the barrier includes an adhesive material. In certain embodiments the adhesive material is selected so as to not interfere with the transparent nature of the barrier material. Any suitable fixing means may be utilized to releasably adhere the barrier material to the control surface of an anesthesia machine. For example, a peelable or pressure sensitive adhesive may be applied to one surface of the barrier. In some forms, the barrier has an inner surface configured to contact a component of the anesthesia machine and an outer touch surface. In some forms, an adhesive is applied uniformly on the inner surface. In other embodiments an adhesive is applied to selected portions of the inner surface for example, at or near the edge of the material. Preferably an adhesive is selected which will not leave a residue, or leaves an easily cleaned residue (e.g. water soluble) upon removal. In certain embodiments the barrier may be constructed so that it clings to the surface to be covered by means of static or surface tension. Examples of suitable adhesives include, but are not limited to, rubber-based adhesives, acrylic adhesives and modified acrylic adhesives, silicone based adhesives, sugar-based adhesives, hydrogel adhesives and/or hydrocolloid adhesives.

In certain embodiments the barriers presently disclosed may be sterile. For example the barrier may be provided in a sealed and sterile packaging. In some forms, the barriers are configured to be sterilized, for example the barriers may comprise a material suitable for sterilizing (e.g. by autoclave, etc.). In some forms, it may not be necessary for the disclosed barriers to be sterilized. In such cases, the disclosed barriers are configured to provide a clean touch surface.

As discussed herein, the present disclosure provides one or more barriers for use with anesthesia equipment. In some forms the disclosed barriers are configured to provide clean touch surfaces. A touch surface as used herein is any surface routinely touched by the operator of an anesthesia machine in order to control the functions of the anesthesia machine. Common touch surfaces include, but are not limited to, control members and touchscreens which are manipulated by a user in order to control the functions of the anesthesia machine.

Touch surface barriers as disclosed herein may be made from any suitable material. In some forms the touch surface barriers are pre-molded so as to fit over specific control members and/or touchscreens having known dimensions. It is also envisioned that touch surface barriers may be provided which are formed from a conformable material such that the barrier can be used over a variety of touch surfaces without requiring pre-molded device specific applications. Conformable materials for use with the presently disclosed barrier can have an adhesive backing as described herein or may contain another suitable form of attachment for example, an elastic or shape memory material. It is also envisioned that the touch surface barrier can be modified for example, by trimming the material to ensure proper fitment.

In some forms, the disclosure provides barriers configured to fit over one or more control members. As used herein, a control member is a portion of the anesthesia machine which is manipulated in order to operate the anesthesia machine. Control members for which barriers may be configured include, but are not limited to, knobs, handles, buttons, levers, dials, switch, valves, keyboards, and/or gauges. Non-limiting examples of barriers provided by the present disclosure include barriers configured for use with vaporizer controls, flow meters, ventilator controls, gas control knobs, flow knobs, and/or pump controls.

In many instances control members on anesthesia machines are configured to communicate information. For example, certain colors and/or shapes are used to indicate the identity of the gas controlled by the control members. In accordance with certain inventive variants the barrier devices disclosed herein are sufficiently translucent so as to allow the operator to determine the color of the underlying control member. In other embodiments the barrier itself is color coded or configured to receive a label indicating the identity of the gas controlled by the underlying control member. In some forms, the barrier is configured to allow the operator to determine the shape of the underlying control member. For example, the barrier may be configured to allow the operator to distinguish between a standardized nitrous control and a standardized oxygen control which have characteristic ridge patterns along their respective sidewalls. In some forms, the shape of the underlying control member is conveyed via the conformability of the barrier material (i.e. the barrier material conforms to the shape of the underlying control member). In some forms, the barrier is pre-molded to conform to the specific shape of the underlying control member and thus adopts a similar shape.

It is also envisioned that certain barrier devices may include a flange member. The flange member is configured to provide a cover for portions of the anesthesia machine which may be inadvertently touched during use of a control member or touch screen, or in which dust and biological material may settle. In some forms, a barrier is provided having an open end and a closed end and wherein a flange member extends outward from the open end such that in use, the flange protects a portion of the anesthesia machine surface near the control member from contact with an operator. In certain embodiments the flange portion is provided without adhesive to allow the flange member to move over the anesthesia machine surface upon manipulation of the control member. It is also envisioned that a flange member may be provided which extends inward towards the open end of the barrier, thus creating a smaller opening. The inwardly extending flange member is configured to prevent dust and/or biological material from accumulating within the crevice formed between the control member and the anesthesia machine. In certain embodiments, control member barriers are provided having both an outwardly extending flange member and an inwardly extending flange member.

The present disclosure further includes barriers configured to cover non-touch surfaces of an anesthesia machine. Non-touch surfaces include all surfaces of the anesthesia machine that are not control members or touch screens (i.e. surfaces used to manipulate the operation of the anesthesia machine). Non-limiting examples of non-touch surfaces include device walls, tubing, and/or surfaces of the anesthesia cart. In some forms, the barriers are configured to surround non-touch surfaces near a touch surface so as to avoid incidental contact with the machine surface. In other embodiments, barriers are configured to cover non-touch surfaces which are routinely manipulated but do not directly affect the operation of the anesthesia machine. Non-limiting examples of such surfaces include handles, wheels, monitor adjustment members, drawers, work surfaces, and/or cabinets. Barriers for non-touch surfaces may be configured as described herein for touch surfaces.

In some forms, the disclosure provides barriers configured to fit over on one or more touch screens. As used herein, a touchscreen is a portion of the anesthesia machine which simultaneously displays information and can be manipulated in order to operate the anesthesia machine. Touchscreen barriers as disclosed herein are translucent so as to allow the operator to monitor the information displayed on the touchscreen. In some forms, a touchscreen barrier is provided which is electro-conductive so as to allow functionality of the underlying touchscreen through the touchscreen barrier. In some forms, the touchscreen barrier is generally planer in order to lie flat against the underlying touchscreen surface. In some forms, a touch screen barrier is configured to adhere to the touchscreen surface as described herein (i.e. static, surface tension, or via an adhesive).

It is also envisioned that a touch screen barrier may be integrated with one or more non-touch barriers and/or control member barriers. For example, a barrier may be provided which includes a touchscreen portion and a non-touch portion configured to cover the anesthesia machine surfaces surrounding the touchscreen. In accordance with certain inventive variants, a barrier may be constructed having one or more openings configured to allow the surface barrier to fit around a control member. In some forms the surface barrier may have varying layer thickness in order to allow manipulation of underlying buttons or controls. For example, in certain embodiments the surface barrier is generally semi-rigid but contains a more conformable portion configured to overlie one or more control members.

In certain embodiments, the disclosure provides for a conformable touchscreen barrier which can be placed and stretched over a touchscreen housing. For example, on one form the present disclosure provides for a translucent and conformable barrier having an open end with an elastic member configured such that the elastic member stretches around the touchscreen housing and secures the conformable barrier over the touchscreen during use and the barrier is then easily removed.

In certain embodiments, the present disclosure provides barriers with adaptations to facilitate placement on an anesthesia machine and/or removal from an anesthesia machine. Such adaptations may be present on any of the barriers described herein. In some forms, a barrier is provided with an adhesive backing, such barriers may further be provided with a protective backing layer to protect the adhesive prior to placement and which can be removed to expose the adhesive. In the case of larger barriers, such protective backing layers can sequentially be removed during placement to ensure proper fitment of the barrier. In certain embodiments the disclosed barrier may contain an adaptation to facilitate removal of the barrier after use. Exemplary removal adaptations include a pull tab or string, and/or weakened (e.g. perforated) portions. In one exemplary embodiment, a touchscreen barrier is provided having a pull tab, the pull tab having an inner surface which is substantially free of adhesive so as to facilitate removal of the touchscreen barrier.

The present disclosure also provides for kits and systems for providing clean touch surfaces on anesthesia equipment. In some forms, the present disclosure provides a kit containing all of the barrier devices necessary to cover the touch surfaces of an anesthesia machine. For example, the present disclosure provides a kit comprising one or more barriers configured to cover a touchscreen and one or more barriers configured to cover a control member. In a preferred embodiment the kit of the present disclosure comprises at least one barrier configured to cover a touchscreen and a plurality of control member barriers sufficient to cover at least the following control members including vaporizer controls, ventilator controls, gas control knobs, and pump controls.

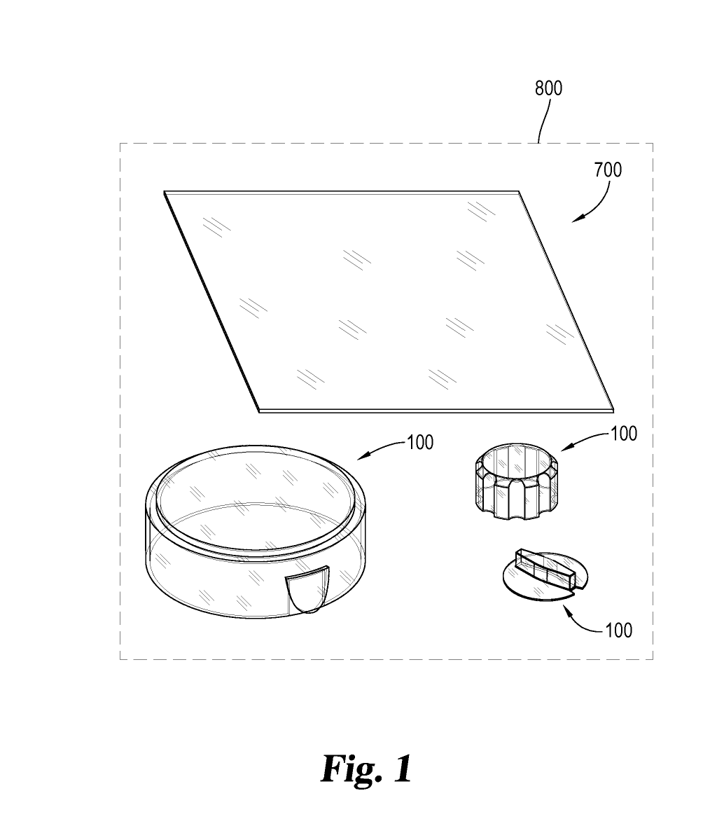

Turning now to the illustrated embodiments, FIG. 1 illustrates one embodiment of a system for providing clean touch surfaces on anesthesia equipment. In the illustrated embodiment the system includes a touchscreen barrier 700, and a plurality of control member barriers 100. In some forms, the system may be provided as a kit 800. In certain embodiments, the kit is provided in the form of a sealed package. It is envisioned that the sealed package may be a sterilized sealed package. The components of the system and/or kit may be customized for the needs of specific anesthesia machines and may include a variety of barriers sufficient to cover the touch surfaces of an anesthesia machine. Other components of a system or kit include, but are not limited to: keyboard covers, writing utensils, and/or non-touch surface barriers.

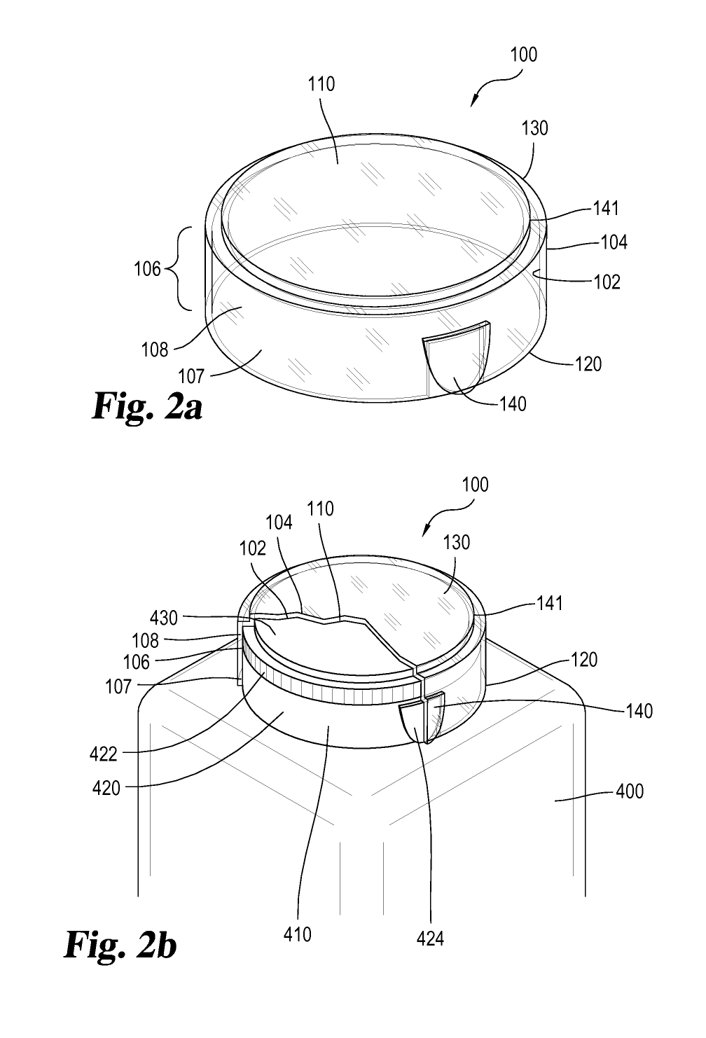

FIG. 2a illustrates one embodiment of a control member barrier. Barrier 100 comprises material 110 and has an inner surface 102 and an outer surface 104. In the illustrated embodiment barrier 100 has a generally cylindrical shape with side wall 106 having a first portion 107 near open end 120 and a second portion 108 near closed end 130. As disclosed herein, in some forms inner surface 102 may further comprise an adhesive. In the illustrative embodiment barrier 100 includes surface adaptations 140 and 141 configured to ensure fitment over one or more features of the control member and/or provide additional functionality as disclosed herein. Surface adaptation 140 comprises part of side wall 106 and when in use may assist in communicating the position of the underlying control member. In the illustrated embodiment surface adaptation 141 comprises part of closed end 130 and is configured to provide a snug fit over the underlying control member when in use.

FIG. 2b illustrates a cutaway view of one embodiment of the control member barrier from FIG. 2a in use over a ventilator control 410. Ventilator 400 includes ventilator control 410 having a side wall 420 and top portion 430. In the illustrated embodiment side wall 420 includes tactile portion 422 configured to facilitate rotation of ventilator control 410 by an operator. In some forms, barrier 100 is sufficiently conformable so as to allow functionality of such tactile portions through the barrier material. In some forms, a premolded barrier is provided with tactile portions molded into the side wall. Molded tactile portions can be present on the inner surface of the control member barrier so as to allow for increased pairing of the control member barrier and the underlying control member. Molded tactile portions may also be present on the outer surface, alone or in conjunction with tactile portions on the inner surface, so as to provide for improved grip on the control member barrier.

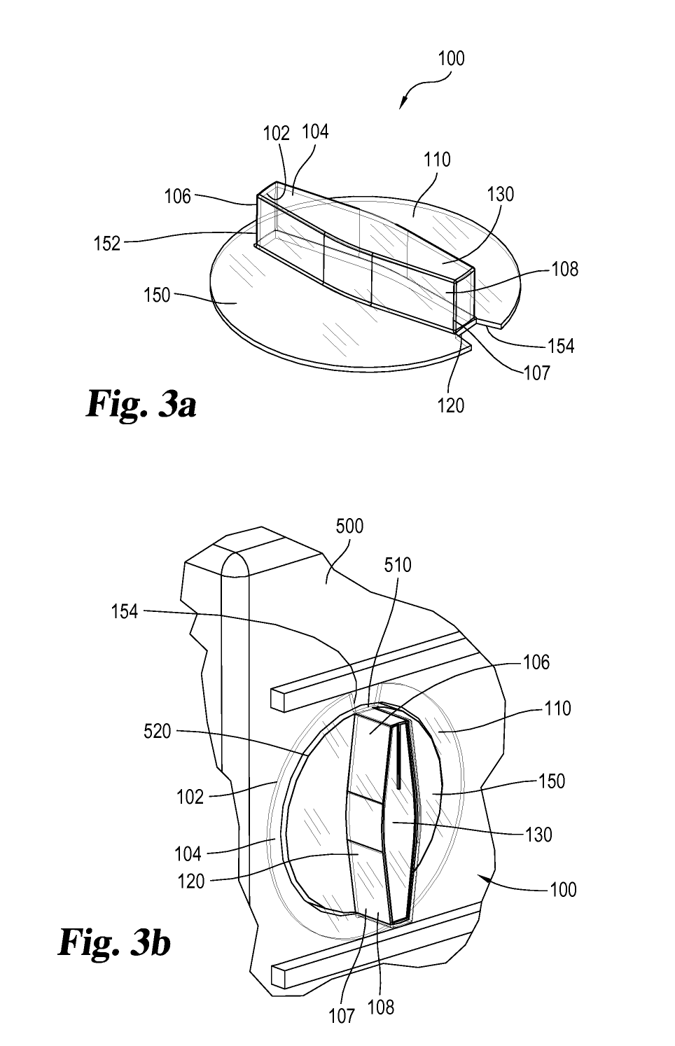

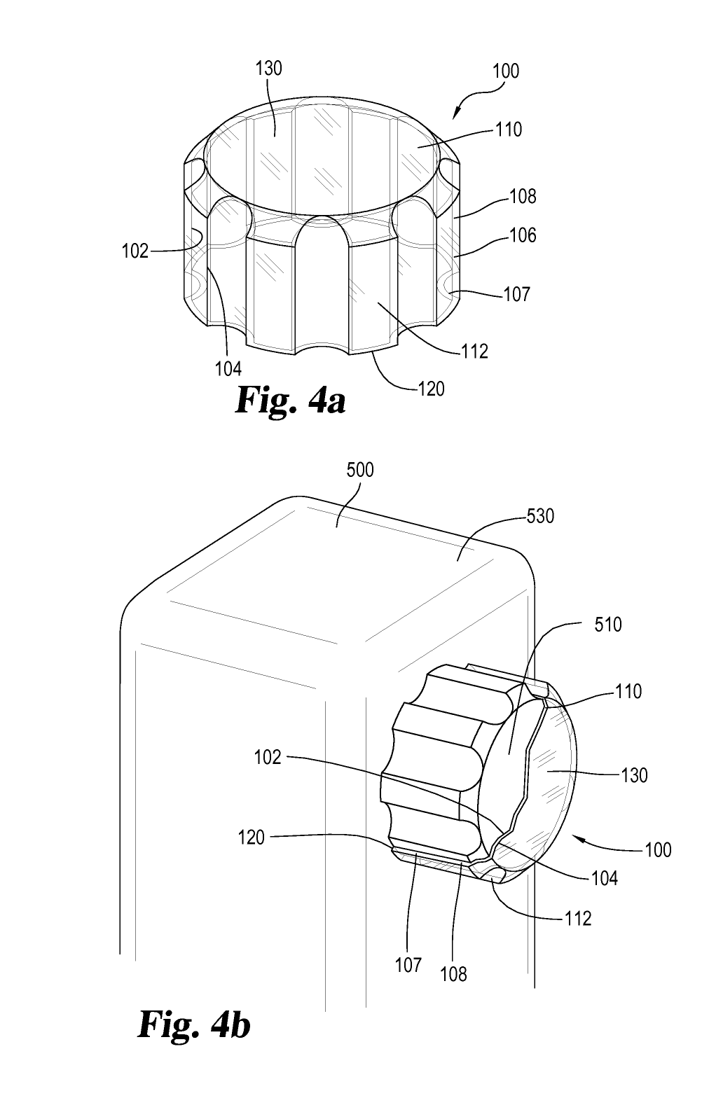

FIG. 3a illustrates another embodiment of a control member barrier. Barrier 100 comprises material 110 and has an inner surface 102 and an outer surface 104. In the illustrated embodiment, barrier 100 is adapted to cover a control member for example, a syringe pump control member. In the illustrated embodiment, barrier 100 includes a flange member 150 and a raised portion 152. Raised portion 152 includes side wall 106, having a first portion 107 near open end 120 and a second portion 108 near closed end 130. Flange member 150 extends away from raised portion 152 and when in use further covers a substantially flat portion of the underlying control member. FIG. 3b illustrates the control member cover of FIG. 3a in use over a control member 510 on anesthesia machine 500. In certain embodiments, flange member 150 includes a cutout portion 154; in other embodiments the flange member may also form a continuous arc around the raised portion. In some forms, cutout portion 154 may aid in placement and/or removal of the barrier material. In the illustrated embodiment, flange member 150 extends beyond the outer edge 520 of the covered control member 510. FIGS. 4a, 4b, and 4c depict further embodiments of control member barriers as disclosed herein.

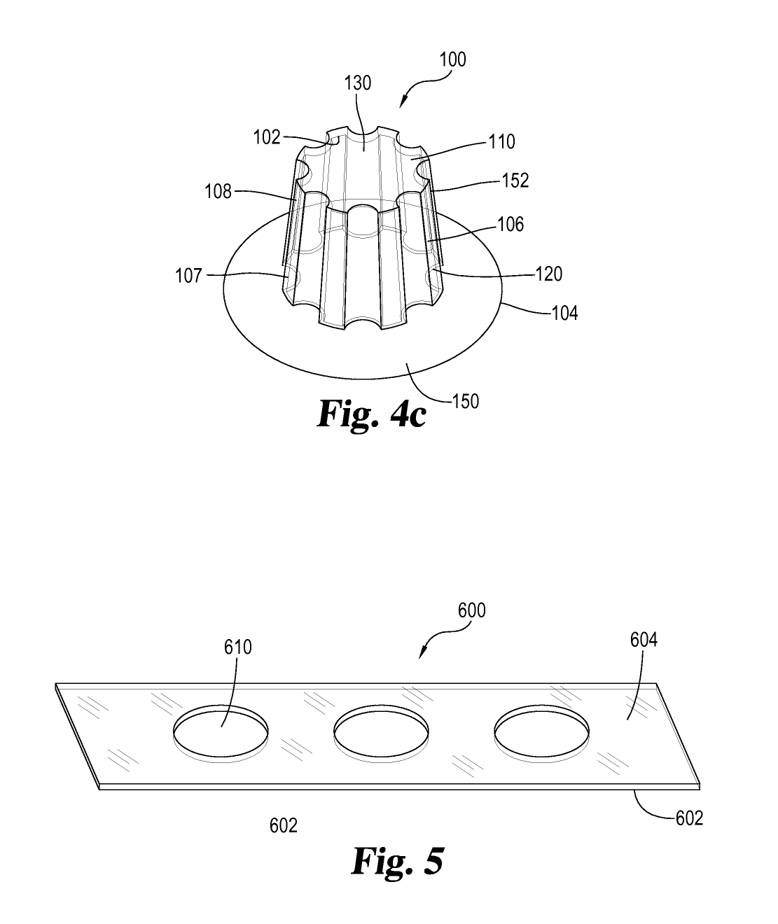

With reference now to FIG. 5, the present disclosure also provides barrier materials for covering non-control surfaces of an anesthesia machine which are commonly contacted during a procedure. For example, the present disclosure provides barrier materials configured to cover device surfaces 530 (see, for example, FIG. 4b) surrounding control members. In the illustrated embodiment, a substantially flat surface barrier 600 is provided having an inner surface 602 and an outer surface 604. Surface barrier 600 includes a plurality of apertures 610 configured to allow a control member to pass through. It is envisioned that in some forms, surface barrier 600 will be used in conjunction with control member barriers disclosed herein.

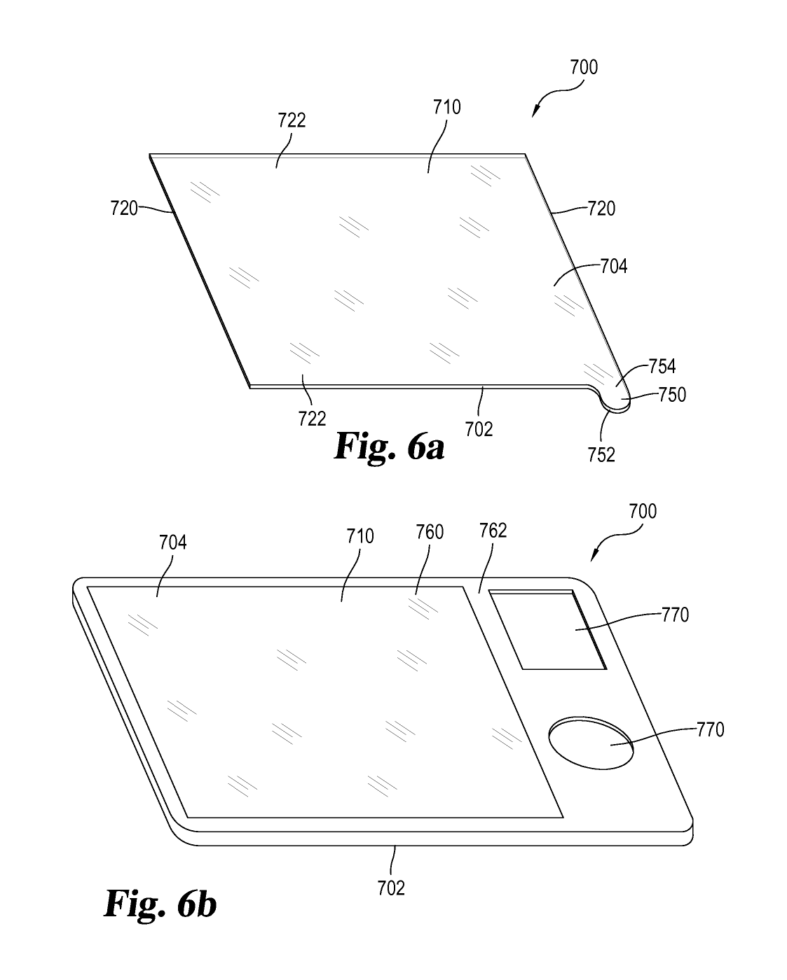

Turning now to a discussion of touchscreen surface barriers, FIG. 6a illustrates one embodiment of a touchscreen barrier 700. Barrier 700 comprises a transparent and electroconductive material 710 having an inner surface 702 and an outer surface 704. In some forms, inner surface 702 is adapted to contact and substantially cover a touchscreen surface of an anesthesia machine. In accordance with some forms of practicing the disclosure, inner surface 702 includes an adhesive layer as described herein. In certain embodiments, the adhesive layer may cover the entire inner surface while in other embodiments, the adhesive layer may comprise adhesive material at or near the vertical edges 720 and/or horizontal edges 722 of barrier 700. The illustrated embodiment further includes tab 750 having tab inner surface 752 and tab outer surface 754. In some forms, tab 750 is configured to facilitate removal of barrier 700. For example, in some forms inner tab surface 752 is presented without an adhesive material to allow a user to grasp and pull tab 750 facilitating removal of barrier 700. Tab 700 may also have an outer surface 754 which in some forms is not translucent or may contain a texturized surface so as to further facilitate locating and removing the barrier material.

FIG. 6b illustrates another embodiment of a touchscreen barrier 700. In the illustrated embodiment, barrier 700 includes a first portion 760 adapted to cover a touchscreen and a second portion 762 adapted to cover a portion of the anesthesia machine surrounding the screen. In the illustrated embodiment, barrier 700 includes cutouts 770 which are adapted to allow a control member (e.g. a dial, button, or lever) to pass through the barrier material. While the illustrated embodiment shows the barrier having cutout portions, it is also envisioned that the barrier will be adapted to allow for functionality of additional control members through the barrier without requiring a cutout portion. For example, all or a portion of the barrier material may comprise a conformable material which allows operation of underlying control members as described herein.

The present disclosure also provides methods of using the system of barriers described herein. In one form, the disclosure provides for a method of using an anesthesia machine comprising the step of applying one or more barriers configured to securely adhere to and substantially cover the control member portion of an anesthesia machine so as to provide a clean touch surface while preserving the functionality of the underlying control member. Such a method may include applying a control member barrier, a touchscreen barrier, and/or a non-touch surface barrier. In some forms, the method includes the step of applying a barrier to one or more ventilator control members, one or more vaporizer control members, and/or one or more syringe pump control members. It is envisioned that the applying step may be accomplished by use of an adhesive as described herein and/or any other suitable technique for securing the barrier material. Non-limiting examples include, but are not limited to, stretching a conformable material or elastic retention device. In certain embodiments, the disclosed method also includes the step of removing the barrier(s) after use of the anesthesia machine.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present disclosure, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

1. A system for providing clean touch surfaces on anesthesia equipment, the system comprising:
one or more touchscreen barriers configured to securely adhere to and substantially cover the touchscreen of an anesthesia machine while being removable after use, said touchscreen barrier also being electroconductive so as to allow full functionality of the underlying touchscreen; and
one or more control member barriers configured to securely adhere to and substantially cover a control member portion of an anesthesia machine while being removable after use.

2. The system of claim 1 wherein said control member barrier is configured to cover a vaporizor control.

3. The system of claim 1 wherein said control member barrier is configured to cover a ventilator control.

4. The system of claim 1 wherein said control member barrier is configured to cover a syringe pump control.

5. The system of claim 1 wherein said control member barrier comprises a transparent material.

6. The system of claim 1 wherein said control member barrier has a color coded region configured to convey the identity of the gas controlled by an underlying control member.

7. The system of claim 1 wherein said control member barrier is premolded and includes one or more tactile portions.

8. The system of claim 1 wherein said touch screen barrier and/or said control member barrier comprise a conformable material.

9. The system of claim 1 wherein said touch screen barrier and/or said control member barrier further comprise one or more adaptations configured to facilitate removal of the barrier.

10. The system of claim 1 wherein said one or more touchscreen barriers and said one or more control member barriers are present in a sealed kit.

11. The system of claim 10 wherein said sealed kit is a sterilized sealed kit.

12. A method of using an anesthesia machine, the anesthesia machine having one or more control members, the method comprising:
applying one or more barriers configured to securely adhere to and substantially cover the control member portion of an anesthesia machine so as to provide a clean touch surface while preserving the functionality of the underlying control member.

13. The method of claim 12 further comprising removing the barrier after use of the control member.

14. A barrier for use with an anesthesia machine, the barrier comprising:
an inner surface configured to contact a portion of the anesthesia machine;
an outer surface configured to be contacted by a user of the anesthesia machine such that the barrier allows full functionality of the covered portion of the anesthesia machine while preventing user contact with the machine.

15. The barrier of claim 14, wherein said barrier has a flange portion encircling a raised portion, the raised portion forming a cavity for receiving a control member of the anesthesia machine.

16. The barrier of claim 14, further comprising an adhesive coating at least a portion of said inner surface.

17. The barrier of claim 14, wherein the barrier is composed of a conformable material such that in use, the barrier conforms to the underlying surface.

18. The barrier of claim 14, wherein the barrier is composed of a premolded material.

19. The barrier of claim 14, wherein the barrier comprises an electroconductive material.

20. The barrier of claim 14, further comprising an adaptation to facilitate removal of the barrier.
